(19) United States
(12) Patent Application Publication
(10) Pub. No.: US 2009/0088707 A1
(43) Pub. Date: Apr. 2, 2009

(54) DRESSING, METHOD AND KIT FOR SKIN CRACK REPAIR

(76) Inventor: Michael Adams, Arvada, CO (US)

Correspondence Address: Michael Adams, 7641 Lewis Court, Arvada, CO, 80005, US

(21) Appl. No.: 12/243,120

(22) Filed: Oct. 1, 2008

Related U.S. Application Data
(60) Provisional application No. 60/976,466, filed on Oct. 1, 2007.

(51) Int. Cl. A61F 13/02

(52) U.S. Cl. 604/307

(57) ABSTRACT

The invention provides a dressing, method and kit components useful for effectively and conveniently treating skin cracks or other skin conditions requiring a moist healing environment.

### CROSS REFERENCE TO RELATED APPLICATION

This application claims the benefit of priority under 35 U.S.C. §119(e) to U.S. Provisional Patent Application Ser. No. 60/976,466 filed Oct. 1, 2007, which is incorporated herein by reference.

### TECHNICAL FIELD

The invention resides in the field of skin treatments and methods of repairing cracked skin.

### BACKGROUND OF INVENTION

Skin cracks occur most commonly on the tips of the fingers and on the heels and elbows. They can also occur anywhere on the body the skin gets excessively dry. As the skin at these locations gets dry, the skin splits or cracks and produces a painful open wound. If untreated these cracks get bigger and more painful, making use of your fingers and/or feet difficult.

There are many ways to treat skin conditions due to dryness. Most current methods to treat skin cracks do not target skin cracks specifically and are thus ineffective in promoting the healing of skin cracks. These products also only keep the skin moisturized for a very short time period, which is typically insufficient to provide any real healing of skin cracks or other conditions reliant on a moist environment for healing. The washing of hands and other activities similar to this also work to remove applied topical moisturizers from the skin allowing the condition to worsen. These topical products may also be greasy and have scents added to them, making them inconvenient or difficult to use in the course of daily activities and/or unappealing or inappropriate to many users.

One method used to heal skin cracks, described in U.S. Pat. No. 5,786,366, is to liberally coat the hands and feet with a moisturizing agent and then cover the affected hand or foot with a glove or sock. The glove or sock minimizes the transfer of the moisturizer to other objects and helps to retain moisturizer on the affected skin. This method is great for moisturizing the entire hand or foot, but does not target skin cracks specifically. As the hands or feet are inserted into the gloves or socks, the moisturizer is actually rubbed off of the fingertips and the heel. Because skin cracks typically appear at the end of the finger or on the heel, the removal of the moisturizer from these areas diminishes the efficacy of this method. Moisturizing with gloves and socks is also impractical for most people because they cannot wear the medicament filled gloves or socks during normal activities. As a result, the use of medicament filled gloves or socks is typically limited to sleeping hours. Many people find this method inconvenient and ineffective for treating skin cracks because it does not provide any therapeutic benefit during activities that actually contribute to excessive skin dryness.

The device described in U.S. Pat. No. 6,410,818 directly targets skin cracks. This device is used to pull the skin crack closed in order for the wound to heal. It does not provide for any medicament application in order to treat pain or infection. This devise would also be difficult to use on skin cracks located around a fingernail.

Skin cracks need both concentrated treatment with a therapeutic agent and a moist healing environment. U.S. Pat. No. 4,812,305 describes a medicine strip with a well for medicine. One problem with this device is that it is designed to allow for air circulation around the wound. This feature renders the device in U.S. Pat. No. 4,812,305 unable to treat skin cracks because it promotes drying of the wound, thus worsening the condition. Air circulation also prevents the medicament used in this device from consistently maintaining body temperature so as to promote absorption and healing. This device is designed to be filled with medicament after it is applied to the wound and it may also be refilled without removal from the skin. The refilling of this device also contributes to wound drying and poor temperature control by repeated exposure to the outside environment. The dressing in U.S. Pat. No. 4,812,305 exerts mild pressure around the wound because of the adhesive strip holding it down. This aspect of the device affects blood flow to the wound impeding healing.

The devise described in U.S. Pat. No. 6,096,943 is designed to protect a wound from external insult and to deliver medication to that wound if needed. Although this device is capable of elongation, it is not designed to bend at the angle necessary to treat a skin crack. Bending this device would cause the bubble to collapse, effectively eliminating the device's ability to cushion a wound. Collapsing of the bubble would also force the unintentional evacuation of the medicament from the bubble.

Coating agents that are brushed onto the skin crack are also available for the treatment of skin wounds. These coating agents are intended for topical application to the wound. The coating agent then solidifies, much like a liquid bandage. This method is effective for simply covering and protecting the wound, but it has very limited healing qualities and has no medicinal or pain relieving effects.

Thus, there remains a need for an effective method of treating skin cracks that forms a moist healing environment and protects the cracked skin from air and pressure to allow healing in the presence of topical medicines.

### SUMMARY OF INVENTION

The present invention provides a device, a method and a kit for promoting the healing of skin cracks and other skin conditions that overcome the problems of the prior art. The device is an occlusive dressing consisting of a doughnut shaped pad, adhesive on one surface of the pad and a waterproof adhesive strip covering the opposite surface of the pad. The dressing provides cushioning for the wound to prevent additional physical trauma and is designed to bend such that it may be used to treat skin cracks around the fingernails or elbows. It also localizes the medicament to the wound, preventing it from inadvertently contaminating other locations. This dressing also does not allow free air circulation. This promotes healing by preventing drying of the skin and subsequent worsening of the wound and/or scaring, and it provides insulation to keep the medicinal contents of the reservoir at body temperature to augment healing.

One aspect of the present invention is a device for promoting the healing of skin cracks. The device is an occlusive dressing that includes a doughnut shaped foam pad with an adhesive strip covering one side of the foam pad. This adhesive covering on one side of the pad forms a reservoir in the center hole of the foam pad. The other side of the foam pad has a pressure-sensitive adhesive layer on the foam pad. The adhesive functions to attach the device to skin. The reservoir defines a volume for at least one medicament to be held in contact with the skin after application of the dressing to the skin of the user. The reservoir may be filled with a medicament before application of the device to the skin. The device is formulated to prevent free air flow between the reservoir and the external environment and to prevent air from contacting the skin. Preferably, the density of the foam pad is chosen to form an occlusive dressing preventing air from circulating through the dressing and from contacting the skin under the dressing.

Preferably, the foam pad is flexible enough to bend over a nail bed or with an elbow or other part of human body. Preferably, the foam pad is also sufficiently compressible to protect the skin crack from tactile pressure.

Another embodiment is a method of treating skin wounds, and particularly skin cracks, that includes filling the pre-formed reservoir within the aforementioned occlusive dressing with medicament and then positioning the filled well over the skin crack. The dressing holds the medicament in contact with the wound and provides an optimal environment for moist wound healing.

In one embodiment of the methods of the invention, an occlusive dressing is used to treat a skin crack. The adhesive dressing includes a doughnut shaped foam pad with an adhesive strip covering one side of the foam pad, and has a pressure-sensitive adhesive layer on one side of the foam pad opposite the side covered by the adhesive. The reservoir of the dressing is filled with a medicament and the device is applied to the skin such that the medicament reservoir is positioned over the skin crack. In one embodiment, pressure is applied to the device to promote adherence to the skin and to secure a medicament within the reservoir of the dressing to the skin. A related embodiment of these methods includes removal of the dressing from the skin and disposal of the dressing after only one application of medicament to the skin. In these methods, the dressing is preferably applied to the skin for a time between about 8 hours and about 12 hours.

Another embodiment of this invention is a kit containing components to treat this condition according to methods of the invention. In one embodiment the kit contains at least the occlusive dressing, a medicament and instructional materials for preparing and applying the occlusive dressing. A preferred embodiment is a kit for promoting the healing of skin cracks containing an occlusive bandage that is a doughnut shaped foam pad with an adhesive strip covering one side of the pad, to form a reservoir in the foam pad, with a pressure-sensitive adhesive layer on the opposite side of the foam pad. The adhesive is chosen to secure the foam pad to the skin with a medicament in the reservoir area. The kit also contains written instructional materials explaining to the user appropriate methods of preparing and applying the occlusive dressing. The written instructions may also explain the effects and preparation of the medicament and describe recommended times and duration of the occlusive dressing.

### BRIEF DESCRIPTION OF DRAWINGS

FIGS. 1-3 show an exploded view of one embodiment of the present invention.

FIGS. 4-6 show methods of using the embodiments of FIGS. 1-3.

### DESCRIPTION OF EMBODIMENTS

The current embodiment of this dressing is configured to apply and hold medicament and other therapeutic agents properly positioned against a skin crack to produce the best therapeutic healing environment.

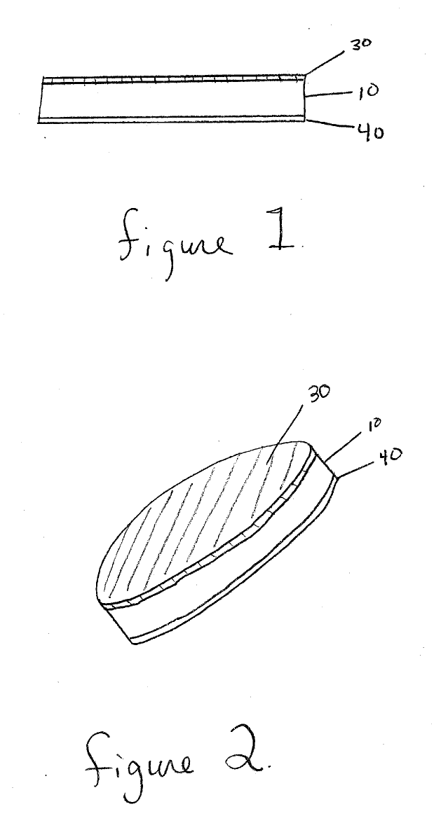

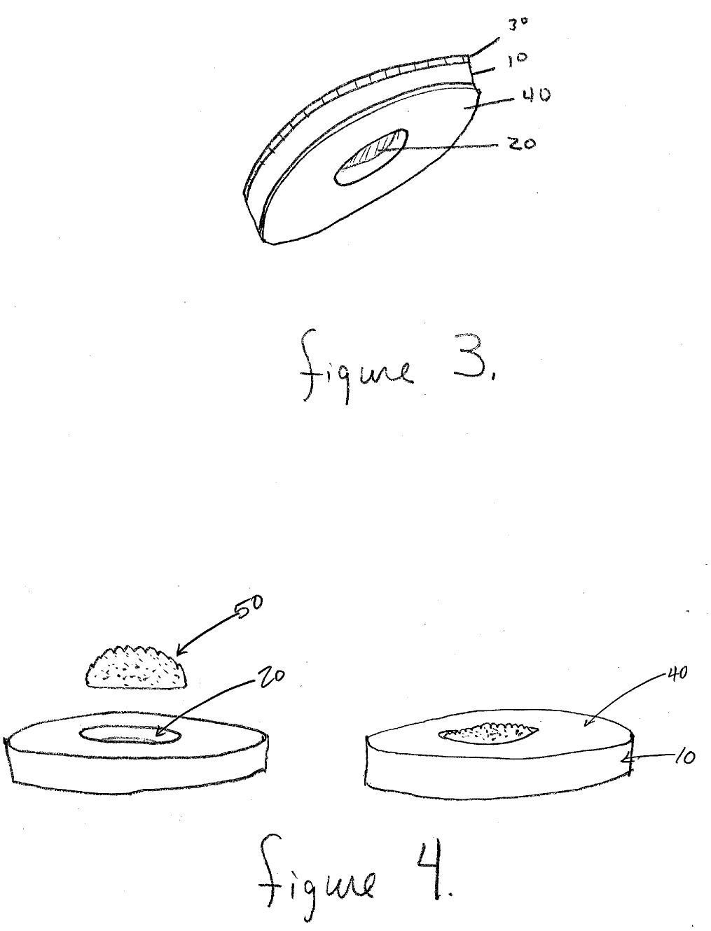

Referring to FIGS. 1-3, this device consists of an adhesive foam, doughnut-shaped pad 10 with an empty reservoir 20 in the middle. One side of the reservoir is closed with a waterproof adhesive strip 30. The other side is coated with a pressure sensitive adhesive 40 with the reservoir open for medicament placement.

The dressing consists of a doughnut-shaped foam pad of variable thickness designed to be pliable enough to bend and flex so as to allow for the dressing to be bent over a fingernail or conform to the movements of an elbow. The pad also mitigates pain by cushioning the wound against tactile insult. One surface of this pad is coated with a hypoallergenic pressure sensitive adhesive designed to hold the pad onto the skin even under the stress of bending. A water resistant strip adheres to the opposite side of the pad. The density of the foam pad is such that air cannot freely move between the dressing reservoir and the external environment. The complete filling of the reservoir also limits airflow by eliminating any air pockets near the skin crack. By limiting airflow around the wound, moisture is retained in proximity to the wound. Skin cracks and other wounds caused by excessive skin dryness need a moist environment in which to maximize healing. The lack of airflow through the dressing serves as insulation to keep the medicament at approximately body temperature to maximize its healing capacity. This device is designed for short-term (about 8-12 hours) use and to be disposed of thereafter. This prevents the development of irritation or hypersensitivity reactions to any components of the dressing. In this embodiment there is no adhesive strip holding down the pad because the pad itself adheres to the skin. Thus, the pad exerts no pressure on the area around the wound. This greatly improves the rate at which the skin wound heals because pressure around the wound impedes blood flow to the wound and slows healing.

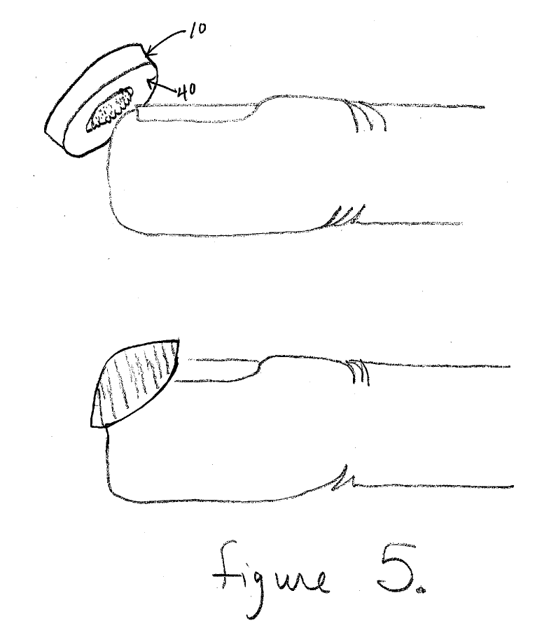

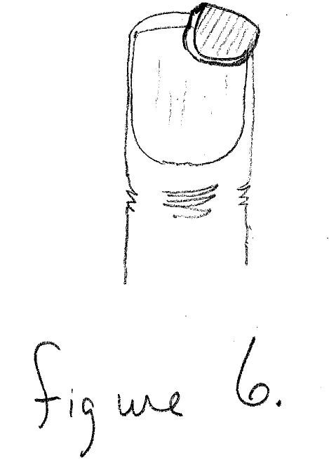

Another embodiment of the present invention is a method to treat skin cracks using the devices of the invention described above. In this method, the reservoir, 20, of the device is first filled with a medicinal composition 50, as shown in FIG. 4. It is then placed topically on the patient's skin such that the medicament filled reservoir within the pad entirely surrounds the skin crack and prevents contact of air to the skin wound, FIG. 5. The adhesive portion of the pad holds the device in place, securing the medicament and reservoir in occlusive contact with the skin crack, FIG. 6. The device is preferably utilized for about 8 to about 12 hours and then discarded after a single use to prevent the infection and contamination often encountered by continuous or multiple uses of a single dressing.

This novel treatment methodology is more convenient than currently existing treatment methods by isolating and concentrating the medicaments needed to effectively treat skin cracks. The medicament is not spread over the entire hand or foot; it is directly applied to the wound. The invention provides intense therapy without spreading the medicament to clothing or other objects the patient touches, reducing the mess and inconvenience of other methods. By filling the space delineated by the adhesive pad and the adhesive strip, an appropriate quantity of medicament is applied that will provide therapy to the skin crack for many hours, helping to rapidly heal the wound. This treatment methodology may be used any time of day without significantly interfering with the patient's normal daily activities.

Several related alternative embodiments of the present inventive methodology may be used to produce a rapid and effective skin crack treatment, including the use of many different therapeutic agents, including but not limited to, topical pain relievers, antibiotics, bleaching agents, antifungal agents and moisturizers. Many different types and strengths of topical pain relievers may be used in the reservoir. These pain relievers may also be applied in different formulations (such as liquids, gels, sprays, etc.) either before or after application of the medicament within the reservoir in the adhesive pad. Different sizes, shapes and colors of adhesive strips may be used to completely, but unobtrusively, cover the affected skin. Adhesive strips may be used with or without absorptive padding.

In one embodiment of the invention, an adhesive strip may be provided with a non-adhesive pad where the pad is held in place on the skin crack by the adhesive strip. Adhesive padding of varying thickness, shapes and reservoir volume may also be used depending upon the location and extent of the skin crack being treated.

Another embodiment of the invention is a device constructed with the medicament pre-loaded in the reservoir, prior to application to the patient's skin wound. This eliminates the necessity of filling the reservoir before application of the bandage to the wound. In this embodiment, the foam pad and adhesive strip are constructed to contain the medicament without significant leakage into the pad or surrounding environment.

One embodiment of the invention is a kit containing components and instructions for treating skin cracks. The kit may contain an adhesive padding, adhesive strip, medicament and instructional materials describing methods of using the kit components. The medicament composition may contain additional therapeutically effective topical pharmaceutical agents useful in preventing, treating or alleviating the discomfort of skin cracks or other skin conditions needing a moist healing environment. The medicament composition may be packaged in manufactured aliquots prepared to be applied and fit easily in the reservoir in the adhesive pad. In another embodiment, these aliquots may be contained within a single dispensing device. As noted above, any one or several of the configurations of adhesive padding and/or adhesive strips, as described above, may be included in the kit of the present invention.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A device for promoting the healing of skin cracks comprising:
a doughnut shaped foam pad;
an adhesive strip covering a first side of the foam pad thereby creating a reservoir in the foam pad; and,
a pressure-sensitive adhesive layer on a second side of the foam pad opposite the first side of the foam pad, wherein the adhesive is sufficient to attach the device to skin.

2. The device of claim 1, wherein the reservoir defines a volume of medicament to be added to the reservoir.

3. The device of claim 1, wherein the reservoir is filled with medicament before application of the device to the skin.

4. The device of claim 1, wherein the device does not allow for free air flow between the reservoir and the external environment.

5. The device of claim 5, wherein the density of the foam pad prevents air from contacting the skin.

6. The device of claim 5, wherein the filling of the reservoir replaces the air within the reservoir with medicament.

7. The device of claim 1, wherein the foam pad is flexible enough to bend over a nail bed or with an elbow or other part of human body.

8. The device of claim 1, wherein the foam pad is compressible to protect the skin crack from tactile pressure.

9. A method for promoting the healing of skin cracks comprising:
a. providing a protective device comprising
a doughnut shaped foam pad;
an adhesive strip covering a first side of the foam pad thereby creating a reservoir in the foam pad; and,
a pressure-sensitive adhesive layer on a second side of the foam pad opposite the first side of the foam pad, wherein the adhesive is sufficient to attach the device to skin;
b. filling the reservoir of the device with medicament;
c. applying the device to the skin such that the medicament reservoir is positioned over a skin crack.

10. The method of claim 9, further comprising applying pressure to the device to promote adherence to skin.

11. The method of claim 9, wherein adhesion of the device to skin secures a medicament within the reservoir.

12. The method of claim 9, further comprising removal of the device from the skin and disposal of the device.

13. The method of claim 9, wherein the device is applied to skin for a time between about 8 hours and about 12 hours.

14. A kit for promoting the healing of skin cracks comprising:
a. an occlusive bandage comprising:
a doughnut shaped foam pad;
an adhesive strip covering a first side of the foam pad thereby creating a reservoir in the foam pad; and,
a pressure-sensitive adhesive layer on a second side of the foam pad opposite the first side of the foam pad, wherein the adhesive is sufficient to attach the device to skin;
b. a medicament composition;
c. written instructional materials.
